(19) United States
(12) Plant Patent Application Publication
(10) Pub. No.: US 2007/0016992 P1
(43) Pub. Date: Jan. 18, 2007

(54) Calla lily plant named 'Picante'

(76) Inventors: Andrew G.F. Warren, Tauranga (NZ); Jacobus P. Spaans, Tauranga (NZ)

Correspondence Address: JONDLE & ASSOCIATES P.C., 858 HAPPY CANYON ROAD SUITE 230, CASTLE ROCK, CO, 80108, US

(21) Appl. No.: 11/183,732

(22) Filed: Jul. 13, 2005

(51) Int. Cl. A01H 5/00

(52) U.S. Cl. PLT/263

(57) ABSTRACT

A calla lily plant particularly distinguished by orange flowers and sagittate leaves is described.

### GENUS AND SPECIES

Zantedeschia spp.

### VARIETY DENOMINATION

‘Picante’

### BACKGROUND OF THE NEW PLANT

The present invention comprises a new and distinct cultivar of calla lily, botanically known as Zantedeschia spp., and hereinafter referred to by the cultivar name ‘Picante’. The new cultivar was asexually reproduced from vegetative cuttings and tissue culture resulting from the cross of the female parent ‘Pot of Gold’ (patent status unknown) and the male parent, ‘Neroli’ (patent status unknown).

‘Picante’ is a product of a planned breeding program intended to create new calla lily plants with orange spathes.

The new cultivar was created in Tauranga, New Zealand and has been asexually reproduced repeatedly by tissue culture in New Zealand. The present invention has been found to retain its distinctive characteristics through successive propagations.

### DESCRIPTION OF PHOTOGRAPHS

This new calla lily plant is illustrated by the accompanying photographs which show the mature inflorescence and foliage of the plant. The colors shown are as true as can be reasonably obtained by conventional photographic procedures.

FIG. 1 shows the mature inflorescence.

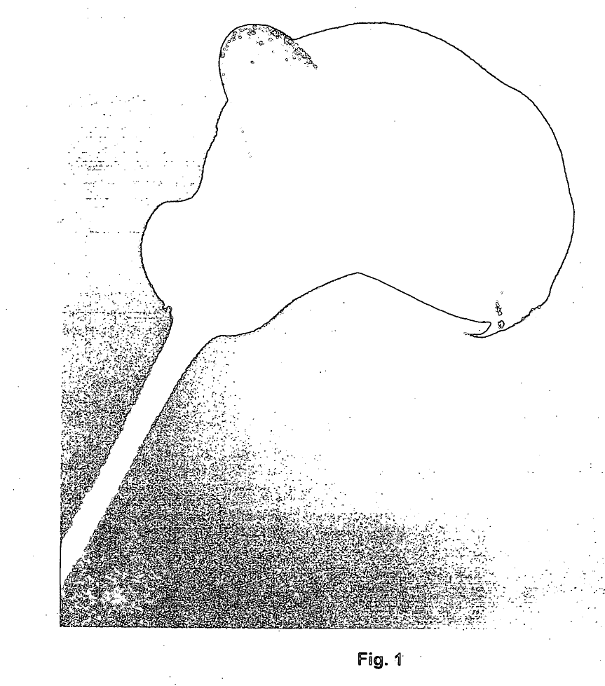

FIG. 2 shows a close-up of the mature inflorescence.

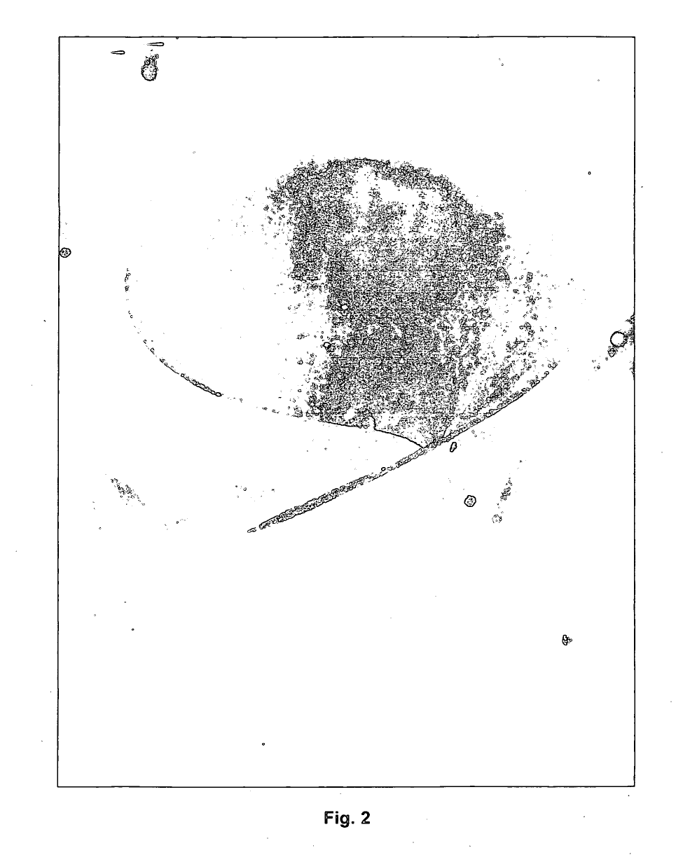

FIG. 3 shows a close-up of the leaf.

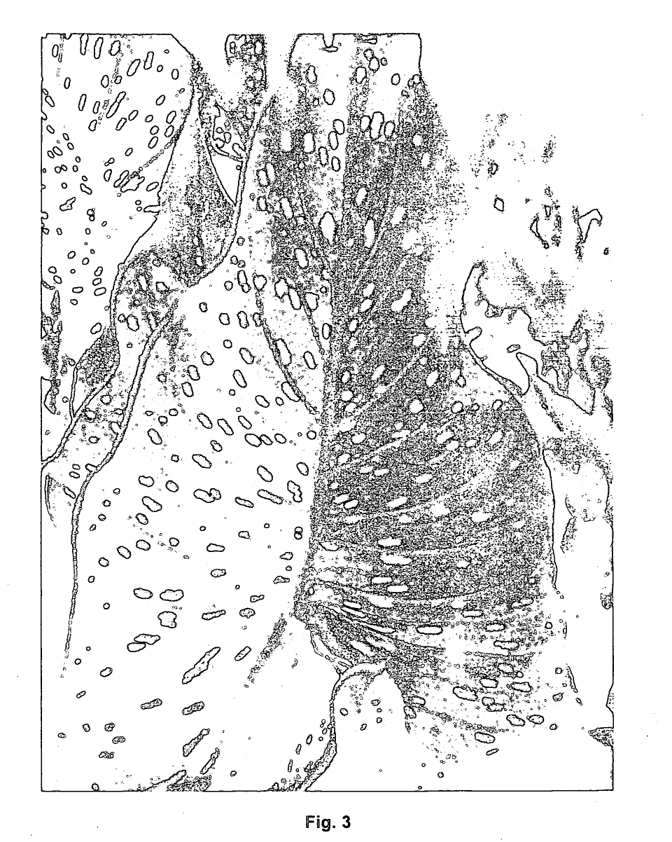

### DESCRIPTION OF THE NEW CULTIVAR

The following detailed description sets forth the distinctive characteristics of ‘Picante’. The data that define these characteristics were collected from asexual reproductions carried out in New Zealand. The plant history was taken on two-year-old plants based on a 3-cm diameter tuber under New Zealand summer conditions. Color references are primarily to the RHS Colour Chart of The Royal Horticultural Society of London (RHS).

### DETAILED BOTANICAL DESCRIPTION

Classification:
   Botanical name.—Zantedeschia spp.
   Common name.—Calla lily.
Size:
   Form.—Erect.
   Height of the leaf canopy above the soil.—45-60 cm.
   Height of top of inflorescence above the soil.—50-65 cm.
   Diameter.—25-40 cm.
   Number of inflorescences per tuber size.—Tuber diameter 3 cm: 2-4 Tuber diameter 4 cm: 3-5 Tuber diameter 5 cm: 5+.
Branches:
   Character.—Strong.
   Color.—Upper: RHS 137C Lower: RHS 137D Base: RHS 155D.
   Average Number.—1-3.
Leaves:
   Size.—Width: 10-14 cm Length: 20-25 cm.
   Shape.—Sagittate.
   Color.—Upper surface: RHS 137B Lower surface: RHS 137C.
   Maculation (leaf spots).—Density: High density Color: RHS 155C.
   Apex.—Acute to apiculate.
   Margin.—Moderately wavy.
   Margin Color.—RHS 144B (yellow-green).
   Veins.—Pinnate.
   Surface texture.—Leathery.
Petiole:
   Length.—20-25 cm.
   Color.—Base: RHS 137D Upper portion: RHS 137C.
Roots:
   Color.—RHS 155D (white).
   Branching.—Not branched, attached to the top of the tuber at apices.
Inflorescence:
   Time to produce a finished flowering plant.—6-10 weeks depending on climate/environment.
   Lastingness of flowers.—2-3 weeks.
   Fragrance.—None.
Flower description:
   Spathe (flower body).—Size: Length: 8-10 cm Width: 5.5-6.5 cm Color: Upper surface: RHS 26A Lower surface: RHS 16B Veins: RHS 26A Shape: Round.
   Spadix (reproductive organs).—Size: Length: 2.0-2.5 cm Diameter: 0.4-0.7 cm Color: Base: RHS 145C Upper: RHS 39A Position relative to spathe: Upright
   Peduncle (flower stem).—Length: 35-55 cm Color: Base: RHS 155A Upper: RHS 144A.
Reproductive organs:
   Location of female organs.—Basal position of the spadix.
   Location of male organs.—Upper position of the spadix.
   Perianth (the floral envelope, calyx and corolla).—Conspicuous.
   Stamens.—Not visible before pollen release.
   Pistil.—Number: 20-30 per spadix Color: RHS 17B.
Fruit:
   Number.—10-20 berries.
   Color.—RHS 145B.
Disease and insect resistance:
   Disease resistance.—No susceptibility to date.
   Pest resistance.—No susceptibility to date.

### Comparison with Known Cultivars

When the instant plant is compared to the female parent ‘Pot of Gold’ (unpatented), ‘Picante’ has orange flowers on a yellow-gold base whereas ‘Pot of Gold’ has solid gold flowers. The flower shape of ‘Picante’ is round, whereas the flower shape of ‘Pot of Gold’ is classic. Additionally, the leaf shape of ‘Picante’ is sagittate, whereas the leaf shape of ‘Pot of Gold’ is cordate.

When ‘Picante’ is compared to the male parent ‘Neroli’ (unpatented), ‘Picante’ has orange flowers on a yellow-gold base whereas ‘Neroli’ has apricot-colored flowers. The flower shape of ‘Picante’ is round, whereas the flower shape of ‘Neroli’ is fluted. Additionally, the leaf shape of ‘Picante’ is sagittate, whereas the leaf shape of ‘Neroli’ is ovate.

I claim:

1. A new and distinct cultivar of calla lily plant as shown and described herein.
